(19) United States
(12) Patent Application Publication
(10) Pub. No.: US 2024/0350825 A1
(43) Pub. Date: Oct. 24, 2024

(54) Light Treatment Device

(71) Applicant: Johann Verheem, Carlsbad, CA (US)

(72) Inventor: Johann Verheem, Carlsbad, CA (US)

(21) Appl. No.: 18/762,632

(22) Filed: Jul. 2, 2024

Related U.S. Application Data
(63) Continuation-in-part of application No. 18/305,331, filed on Apr. 22, 2023.
Application No. 18/305,331 is a continuation of application No. 17/029,944, filed on Sep. 23, 2020, now Pat. No. 11,666,776.
Application No. 17/029,944 is a continuation-in-part of application No. 16/819,016, filed on Mar. 13, 2020, now abandoned.
(63) Continuation of application No. 16/819,016, filed on Mar. 13, 2020, now abandoned.
(60) Provisional application No. 62/878,150, filed on Jul. 24, 2019.
(60) Provisional application No. 63/008,866, filed on Apr. 13, 2020.
(60) Provisional application No. 63/010,681, filed on Apr. 15, 2020.
(60) Provisional application No. 62/878,150, filed on Jul. 24, 2019.
(60) Provisional application No. 62/818,180, filed on Mar. 14, 2019.

(51) Int. Cl. A61N 5/06; A61M 37/00

(52) U.S. Cl. CPC A61N 5/0616; A61M 37/00; A61N 2005/0629; A61N 2005/063; A61N 2005/0632; A61N 2005/0644

(57) ABSTRACT

A device for skin treatment includes a body and a rotatable head affixed thereto, with the head containing a plurality of rotationally or radially symmetric regions of resilient and/or rigid bristles and/or protrusions, and/or regions having a wavy surface. The head can include transparent or translucent regions to transmit light from light elements such as LEDs on the housing through the head and onto skin while in use. The heads may be partially or completely metallic to facilitate application of heat, cold, and/or galvanic currents to the skin. The device can also be configured to dispense a lotion or other substance through the head.

### CROSS-REFERENCE TO RELATED APPLICATIONS

This application is a continuation-in-part of U.S. application Ser. No. 18/305,331 filed on Apr. 22, 2023, which is a continuation of U.S. application Ser. No. 17/029,944 filed on Sep. 23, 2020 and issued as U.S. Pat. No. 11,666,776 on Jun. 6, 2023, which is a continuation-in-part of U.S. application Ser. No. 16/819,016 filed on Mar. 13, 2020 and which also claims the benefit of U.S. provisional application No. 62/878,150 filed on Jul. 24, 2019, U.S. provisional application No. 63/008,866 filed on Apr. 13, 2020, and U.S. provisional application No. 63/010,681 filed on Apr. 15, 2020; and it is also a continuation-in-part of U.S. application Ser. No. 16/819,016 filed on Mar. 13, 2020, which claims the benefit of U.S. provisional application No. 62/878,150 filed on Jul. 24, 2019, and U.S. provisional application No. 62/818,180 filed on Mar. 14, 2019.

### FIELD OF THE DISCLOSURE

The present disclosure relates generally to devices that are used to apply mechanical manipulation, light treatment, substances, and/or electrical current to skin and other tissues to improve the quality and/or appearance of such tissues.

### BACKGROUND INFORMATION

Cleansing, sanitizing and treating the skin is a common part of many people's daily routines. Many different types of skin creams, lotions, soaps and medications are applied to the skin in order to treat or prevent aging, affect wrinkles, reduce discoloration, pigmentation and age spots and eliminate medical skin conditions such as acne and sanitize or cleanse the skin from contaminants, viruses and bacteria. Skin care and hair care can also include treatment of the human scalp. In addition, nails are often treated with creams, lotions and decorated with paints and other soluble ingredients.

Applying physical force through brushing, scrubbing, wiping and massaging the skin aim to clean and sanitize the surface of the skin, remove dead skin cells and impurities and stimulate the skin to produce new skin cells and repair itself. Adding lotion, creams and medications to a skin care regiment enhances the beneficial effect of physically cleaning and massaging the skin.

Phototherapy and the use of various different light sources, including laser light, LED light sources, and incandescent light sources (including, e.g., halogen light sources) have also been used to provide cosmetic and therapeutic benefits to human skin, hair and nails. Various forms of light have been used to enhance sanitization of surfaces and to treat human skin, hair and nails. Light of different frequencies can be emitted from various light sources onto the skin, which can support production of collagen, increase blood flow, kill viruses and bacteria and reduce inflammation. These light frequencies include, but are not limited to, ultraviolet light, near infrared light, infrared light, and blue light. Phototherapy can also be used outside of the field of skin care to provide cleaning and sanitization benefits.

Hot and cold materials placed on the skin have also been demonstrated to have certain medical, cosmetic and therapeutic benefits. In addition, mechanical vibrations, mild currents from an electric source, and sonic waves have also been demonstrated to provide such benefits.

Accordingly, it may be desirable to provide a convenient apparatus that can provide one or more of such treatments to skin and other tissues, including certain combinations of such treatments.

### SUMMARY OF THE DISCLOSURE

The following simplified summary is provided in order to provide a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented later.

Embodiments of the disclosure can provide a device for treating or affecting skin tissue and other external body parts (such as a scalp) that includes a housing that can be held in the hand. A treatment head can be rotatably affixed to the device, e.g. via a protruding shaft, and may be detachable, and a motor can be provided in the device to rotate the head at one or more desired speeds. In some embodiments, a rotatable disc may be affixed to the shaft, and a removable/replaceable disc cover can be affixed to the disc to provide a rotating surface that affects the skin. This configuration allows for easier replacement of the treatment head between users of the device and/or switching of heads with different surfaces for different treatments, and may be economical for providing heads that are disposable after use.

One or more light sources, such as LEDs, can be provided on the housing below the rotatable head, and the head can have one or more transparent or translucent sections that allow light from the light sources to shine therethrough and onto the skin while the device is being used. The LEDs can be configured to emit one or more colors of light depending on the desired effect. Such light colors include red, blue, infrared, and UV light. In some embodiments, a switch and/or sensor can be provided on the device to detect which type of head is affixed thereto and select one or more light colors accordingly. The light colors may also be selectable by a user, e.g., using another switch or selector on the device.

The device can include a microprocessor and certain electronic and electrical components to control its operation. Such components can include switches to control power to the device, selecting light colors, selecting rotational speeds, a timer to keep the device powered for predetermined time periods, and the like. The device can also include a sensor that detects proximity of the head to the skin or another surface, and can be configured to cut off power to the rotating head and/or lights when the head is not proximal to a surface. This can provide safety benefits by powering down aspects of the device when the head is not close to or in contact with skin or another surface.

The device can also be provided with a chamber that contains an ingredient or substance, such as a lotion, salve, or other liquid, that may be dispensed onto the skin while the device is being used. The substance may be dispensed through a channel provided through the rotating shaft, where a corresponding opening is provided in the head and/or disc and disc cover, or by other nozzles or orifices in the device. Dispensing of the substance can be controlled by a small pump within the device, by compressing a portion of the chamber that has flexible walls, or by other means.

In some embodiments the heads (or disc cover) may have a plurality of soft, resilient, and/or firm/hard bristles or protrusions on it that are configured to interact with the skin surface when the head or disc cover rotates. These protrusions may have the shape of nubs, vertical cylinders, elongated wipers, round or ovoid protrusions or objects, or combinations thereof. Soft or resilient protrusions and rigid protrusions may be combined on a single head. Such protrusions can be provided in rotationally symmetric or radially symmetric configurations, e.g., in concentric circular or ring-shaped regions on the head or disc cover. In some embodiments, the head or disc cover may include transparent or translucent regions between these regions to allow light sources provided on the device to shine therethrough and onto the skin being treated.

In some embodiments, the head or disc cover can include one or more rotationally or radially symmetric regions that is formed of a rigid material having a wavy surface, to provide massaging or vibrational tissue stimulation when the rotating head/cover contacts the skin. In certain embodiments, these wavy regions (or the entire head/cover) can be formed at least in part of a metallic material, which can effectively transfer heat or cold to the tissue. Heating can be achieved, e.g., by conducting heat through the head from light elements or another type of heat-producing arrangement provided in the body of the device (e.g. a resistive heating element and circuit), providing resistive heating elements within the head together with an electrical connection between the body of the device and the rotating head, preheating the head, e.g. by immersing it in hot water, or providing chambers within the head that can contain a heated material. Similarly, cooling can be provided by precooling the head in a refrigerator or freezer, by providing chambers within the head that can contain a cooled material, or by providing a cooling arrangement such as a thermoelectric cooling element within or affixed to the head.

In still further embodiments, a partially or fully metallic head or disc cover can be used to introduced galvanic currents into the skin while using the device. This can be achieved by providing two or more electrically isolated metallic regions in the head, which are in electrical contact with a current or voltage source contained in the device. The electrical connection can be configured, e.g., as brush contacts provided on the head that are in electrical communication with electrical contacts on the rotating shaft, allowing electrical power to be supplied to the metallic regions while the head rotates. The metallic regions can be, e.g., separate wavy rings provided on the head, and/or separate metallic sections of a single wavy ring that are separated by an electrically insulating material.

### BRIEF DESCRIPTION OF THE DRAWINGS

Further objects, features and advantages of the disclosure will become apparent from the following detailed description taken in conjunction with the accompanying figures showing illustrative examples, results and/or features of the exemplary embodiments of the present disclosure, in which:

FIG. 1 is a partially exploded perspective view of a skin treatment device according to certain embodiments of the disclosure;

FIG. 2 is a front perspective view of the device shown in FIG. 1;

FIG. 3 is a front perspective view of a skin treatment device according to further embodiments of the disclosure;

FIG. 4 is a cross-sectional view of the device shown in FIG. 1;

FIG. 5 is a block diagram of representative elements that can be used to operate and control the device;

FIG. 6A shows an exemplary applicator disc with bristles and a transparent/translucent base that can be used with certain embodiments of the disclosure;

FIG. 6B shows an exemplary applicator disc with bristles and transparent/translucent regions between them to facilitate light transmission therethrough;

FIG. 6C shows another exemplary applicator disc with transparent/translucent regions to facilitate light transmission therethrough;

FIG. 7 illustrates another representative device configuration in accordance with certain embodiments of the disclosure;

FIG. 8 shows an exemplary applicator disc with rings of bristles and transparent/translucent regions between them to facilitate light transmission therethrough;

FIG. 9A shows an exemplary applicator disc with rings of a wavy metallic material and transparent/translucent regions between them to facilitate light transmission therethrough;

FIG. 9B is a side view of the head shown in FIG. 9A;

FIG. 9C is a perspective view of the head shown in FIG. 9A;

FIG. 10A shows an exemplary applicator disc with rotationally symmetric areas of bristles and rigid ovoidal protrusions, with transparent/translucent regions between them to facilitate light transmission therethrough;

FIG. 10B shows an exemplary applicator disc with rotationally symmetric areas of bristles and rigid round protrusions, with transparent/translucent regions between them to facilitate light transmission therethrough;

FIG. 11 provides a view of a squeezable tube with an attached applicator that contains a cavity through which the ingredient in the tube can be dispensed as well as one or more sources of light;

FIG. 12 provides and additional view of a tube that contains an ingredient and an applicator cap that also contains one of more light sources that are attached to the applicator cap;

FIG. 13 is a perspective view of a rigid or semi rigid tube with an applicator cap attached on one end, in which one or more light sources are embedded or attached to, as well as a rotatable activator that will squeeze the ingredient embedded in the rigid or semi right tube out through the applicator; and

FIG. 14 shows a perspective view of an embodiment of the device where the applicator is a rotating brush that moves where the ingredient is housed within the body of the brush and where the light source is embedded in between the bristles of the device.

The various embodiments of the disclosure are described herein with reference to the figures, where like reference numbers indicate identical or functionally similar elements. Further features and advantages of the disclosure as well as the structure and operation of various embodiments of the present disclosure are described in detail below with reference to the accompanying drawings. To the extent that the present disclosure does reference the figures, it is done so in connection with the illustrative embodiments and is not limited by the particular embodiments illustrated in the figures. It is intended that changes and modifications can be made to the described embodiments without departing from the true scope and spirit of the present disclosure.

### DETAILED DESCRIPTION

The embodiments set forth below represent the necessary information to enable those skilled in the art to practice the embodiments and illustrate the best mode of practicing the embodiments. Upon reading the following description in light of the accompanying figures, those skilled in the art will understand the concepts of the disclosure and will recognize applications of these concepts that are not particularly addressed herein. It should be understood that these concepts and applications fall within the scope of the disclosure and the accompanying embodiments.

The purpose of the terminology used herein is only for describing embodiments and is not intended to limit the scope of the disclosure. Unless defined otherwise, all terms of art, notations and other scientific terms or terminology used herein have the same meaning as is commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art. All patents, applications, published applications and other publications referred to herein are incorporated by reference in their entirety. If a definition set forth in this section is contrary to or otherwise inconsistent with a definition set forth in the patents, application, published applications and other publications that are herein incorporated by reference, the definition set forth in this section prevails over the definition that is incorporated herein by reference.

Unless otherwise specified herein, the features of the presently disclosed solution may be economically molded or assembled by using one or more distinct parts and associated components which may be assembled together for removable or integral application.

Where context permits, words using singular or plural form may also include the plural or singular form, respectively. As used herein, “a” or “an” means “at least one” or “one or more.” As used herein, the term “user”, “subject”, “end-user” or the like is not limited to a specific entity or person. For example, the term “user” may refer to a person who uses the systems and methods described herein, and frequently may be a technician. However, this term is not limited to end users or technicians and thus encompasses a variety of persons or entities who can use the disclosed systems and methods.

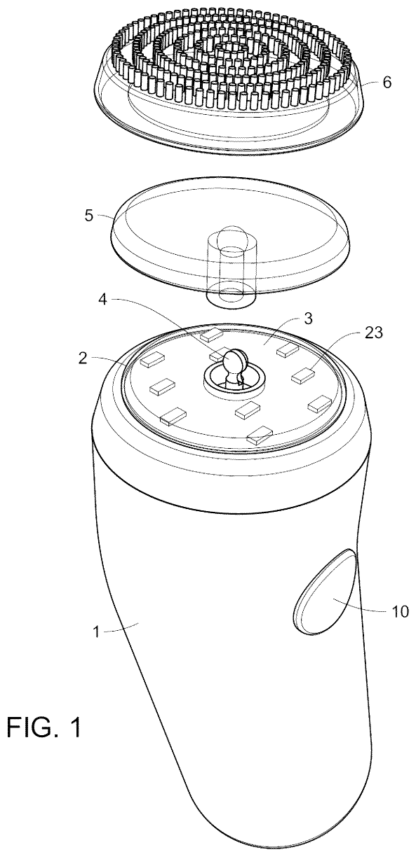

FIG. 1 is a partially exploded perspective view of a representative embodiment of the light therapy device when viewed from the head of the device, which comprises a housing [1] and, at one end of the housing [1], an optional solid, transparent or translucent head plate [2] integral with the housing. The housing [1] is essentially the handle of the device, and to that end can be sized for holding in the hand, such that the head of the device can be controlled, pointed and directed as intended by a user. The housing [1] may comprise a handle or grip in a wide variety of sizes and shapes without limitation. Secured within housing [1] behind transparent or translucent head plate [2] is a panel [3] comprising one or more light sources [23] whereby the one or more light sources [23] can shine through the head plate [2] to provide phototherapy benefits.

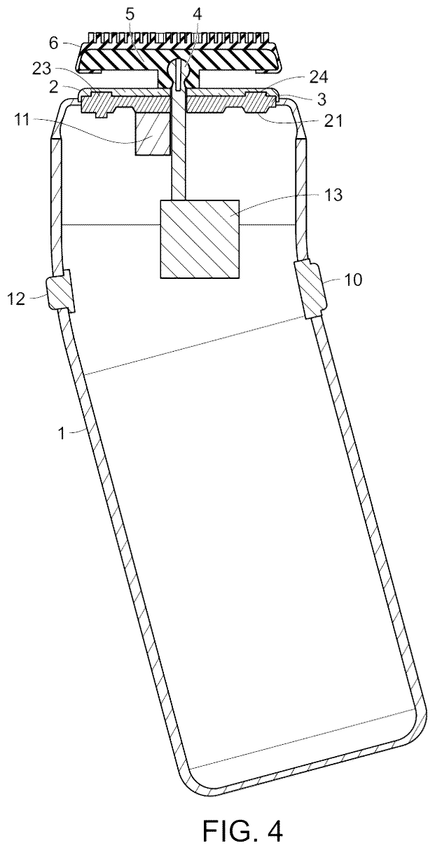

The exemplary apparatus of FIG. 1 can also include an applicator disc [5] that is affixed over the head plate [2]. The applicator disc [5] can also be transparent or translucent to allow the light sources [23] to shine through transparent or translucent portions of the disc cover [6], which may be affixed to the applicator disc [5] and configured to contact the skin or other parts of a subject while the device is in use. The applicator disc [5] (and associated disc cover [6]) may be operatively coupled to a motor [11] secured within housing [1] (see FIG. 4) via a shaft [4] such that applicator disc [5] and disc cover [6] can rotate while the panel [3] with one or more light sources [23] remains fixed. A switch [10] may be included on the housing [1] of the device to turn the device on and off.

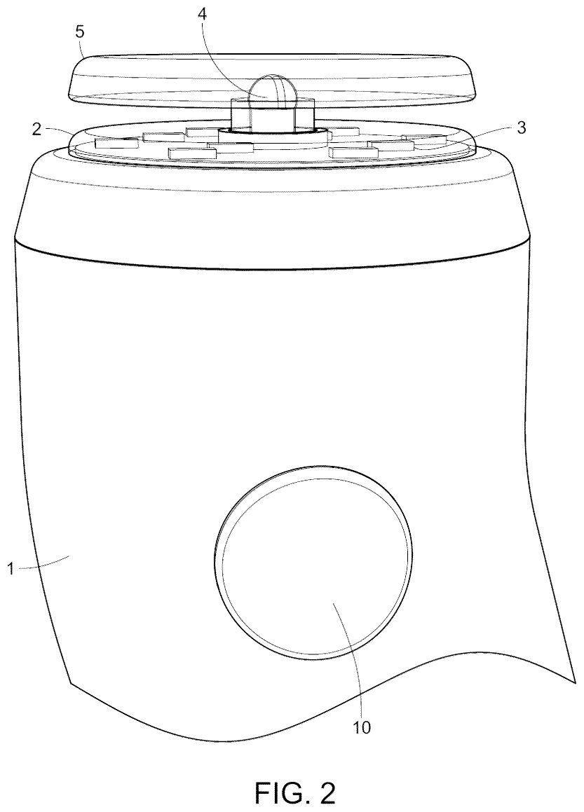

FIG. 2 is a side perspective view of a representative embodiment of the device. An applicator disc [5] is attachable to the device over the head plate [2] via the shaft [4], the applicator disc [5] being substantially transparent or translucent such that the light sources when shining through the head plate [2] also shine through the applicator disc [5]. The applicator disc [5] may be a fully transparent or translucent disc, comprising transparent or translucent surface elements. Alternatively, the applicator disc [5] may comprise one or more transparent or translucent sections that allow light from the light sources [23] to shine therethrough.

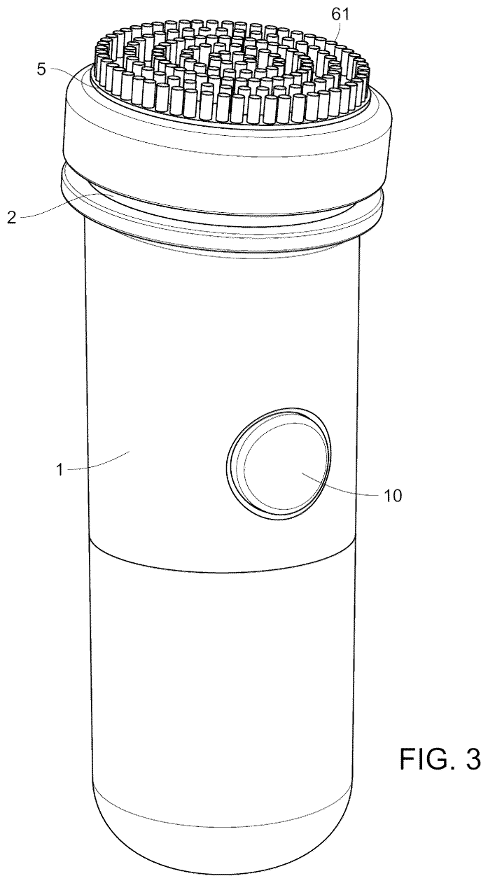

FIG. 3 is a perspective view of a representative embodiment of the light therapy device having bristles [61] incorporated into an applicator disc [5] having a transparent or translucent base. In this embodiment, the bristles [61] are incorporated as part of the applicator disc [5]. For example, as shown in FIG. 3, the bristles [61] can be arranged in concentric rings directly on the applicator disc [5], with openings or transparent/translucent sections between the concentric rings of bristles [61] to allow light from light sources [23] to shine through them and onto a subject. Alternatively, as shown in FIG. 1, such bristles may be provided in a similar configuration as part of a removable disc cover [6], again with transparent, translucent, or open regions between the rings of bristles to allow light to shine through them.

FIG. 4 is a cross-sectional view of a representative embodiment of the light therapy device. Applicator disc [5] may be operatively coupled to a motor [11] secured within housing [1] such that applicator disc [5] is operable to rotate while the panel [3] with one or more light sources [23] remains fixed. For example, applicator disc [5] may be attachable to one end of a shaft [4] operatively coupled to motor [11] within the device. The shaft [4] may transect the head plate [2], for instance protruding through an aperture in the head plate [2]. By rotating the shaft, applicator disc [5] may rotate while the rest of the device, including panel [3] including light sources [23], and head plate [2], remain fixed. The shaft may create a space between applicator disc [5] and head plate [2], facilitating access and movability to applicator disc [5]. For instance, the applicator disc [5] can be made disposable, or can be configured to accept disposable attachments or attachments with different types of operating surfaces, such as the disc cover [6] shown in FIG. 1.

The device may be operable to dispense an ingredient so that simultaneously, the light reaches the ingredient as well as the user's skin, hair, scalp or nails, or other applied surface. The light can impact the ingredient, so that chemical processes or alterations in the ingredient are activated. The light source can also impact the user's skin, scalp or nails, by increasing blood flow, in the case of certain red frequencies of light, or by killing bacteria or viruses on the surface of the skin, such is the case with certain blue frequencies of light. The light can also act as a source of heat that melts the ingredient when dispensed through the applicator, or that heats up either the ingredient, the skin, hair or nails of the user, or both the ingredient and part of the housing in order to provide therapeutic or cosmetic benefits.

For example, the housing [1] may incorporate a chamber [13] to hold said ingredient, which may be transmitted through shaft [4], which can be hollow or have one or more channels provided therethrough, and/or through openings in applicator disc [5] and optional disc cover [6]. Although not illustrated in FIG. 4, various dispensing arrangements can be used to dispense an ingredient from the chamber [13] through the shaft [4] and applicator disc [5] (and optional disc cover [6] if present) while the device is being used. For example, the chamber [13] can be formed at least in part with a flexible or resilient material, and a button, lever, or other engagement component can be provided on the housing [1] that can be pressed against the resilient chamber [13] to force an ingredient contained therein through the shaft [4] and other components and onto a body part of the user. Alternatively, a small pump can be provided within the housing [1] and configured to propel the ingredient from the chamber [13] and through the shaft [4] and other components during use of the device when the pump is activated, e.g. by a switch provided on the housing [1]. In other embodiments, similar to those described herein below, a portion of the housing [1] can be resilient and in contact with the resilient chamber [13], such that squeezing a region of the housing [1] can apply pressure to the chamber [13] and force an ingredient out of it.

Applicator disc [5] and/or disc cover [6] may further comprise a chamber into which various elements such as hot or cold gel containers or ice can be inserted, for instance, such that the surface covering can provide temperature specific benefits to the skin, or said chamber may contain liquid ingredients such as skin creams, lotions, medication, sanitizers and soaps that are dispensed through an opening to the skin of the user. Applicator disc [5] and/or disc cover [6] can be made from a material that retains heat or cold. Applicator disc [5] or disc cover [6] may be impregnated with a material so that it deposits the ingredient to the surface being cleaned.

The panel [3] may comprise a microcontroller [21] for operating the light sources [23], such as LEDs. In other embodiments, the microcontroller [21] and other electronic components described herein may be located within the housing [1]. The microcontroller [21] may be programmable to control the wavelength (and thus frequency) of light waves emitted by the LEDs [23], since different wavelengths of light have different effects in the treatment of various conditions. For instance, blue light wavelengths (e.g., wavelengths between about 400-500 nm) are known as an effective treatment for acne, while infrared wavelengths (e.g., near-infrared wavelengths between about 700-1000 nm) can be provided to stimulate blood circulation and promote wound healing and muscle recovery. Red wavelengths (e.g., wavelengths between about 630-700 nm) can be beneficial for skin rejuvenation. It should be noted that the device is readily adaptable to different uses other than skin treatments, for example, light sources [23] may be configured to emit UV light, which may be used on various articles for sterilization. In some embodiments, light sources [23] can be provided that emit different wavelengths of light to combine or enhance the exemplary effects above.

A switch [10] may be included on the housing of the device to turn the device on and off, and a power source [12] may be included in or on the housing [1] of the device to provide power. Various power sources [12] such as, e.g., batteries or other AC or DC power sources can be implemented. Batteries may be provided within the housing [1], and can be replaceable, or rechargeable through electrical induction or through a port and cable connecting the device to an external power source. In some embodiments, the device can be powered directly by an external power source, such as an electrical outlet or external battery pack, via an appropriate wire or cable.

The panel [3] may additionally comprise a sensor [24], for example on its outer surface, such that the light sources [23] are only activated when the translucent head plate [2] is proximal to or pressed against the skin or other body part of the user, thereby preventing light activation or light shining into the eye of the user. The sensor [24] can also be used to prevent activation of the device (e.g., motor activation and spinning of the applicator disc [5] and optional disc cover [6]) if the device is not adjacent to or pressed onto the skin or other body part of the user.

The panel [3] may be an electronic circuit board comprising one or more light sources such as, e.g., light-emitting diodes (LEDs) [23] on its outer surface, i.e., the surface facing the translucent head plate [2] and/or applicator disc [5].

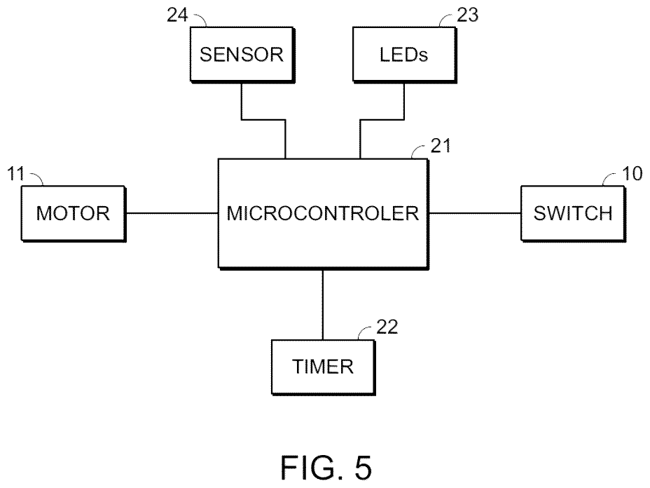

FIG. 5 is a block diagram of certain components that may be used for control and operation of the device. These components include a motor [11] used to spin the applicator disc[5] when activated, a sensor [24] as described herein, and one or more switches [10] that can be used to activate, adjust, or deactivate any of the electrical components.

The transparent or translucent head plate [2] can be assembled integral with the housing [1] of the device and may be planar or it may have other surface topology. The translucent or transparent head plate [2] and/or translucent or transparent applicator disc [5] may integrate lenses to further focus and/or direct the light sources [23].

Different light frequencies, or no light at all, may be optimal for different applicator discs [5] and/or disc covers [6]. Input from a sensor [24] and/or switch [10] may be applied to select the frequency/wavelength of lights emitted by the light sources [23]. a sensor [24] and/or switch [10] may also be provided to detect the type of applicator disc [5] and/or disc cover [6] attached to the housing [1], which may trigger a selected frequency of light emitted by the light sources [23]. For instance, a sensor [24] may detect applicator discs [5] and/or disc covers [6] of selected colors that can be recognized by the sensor [24], each of which may be assigned to a selected frequency of light. As another example, the applicator disc [5] may be attachable to the device via insertion plugs of selected shapes or lengths, or it may have specific protrusions or the like, such that a switch [10] and/or sensor [24] may detect the selected shape or length of the applicator disc insertion plug or protrusions, which may be assigned to one or more pre-selected frequencies/wavelengths of light. In this manner, interchangeable applicator discs [5] and/or disc covers [6] may activate different frequencies of light, or no light at all.

A timer [22] may be operatively coupled to the LEDs [23] such that light is applied for a prescribed amount of time. The panel [3] or housing [1] of the device may additionally comprise a sonic emitter, such as an ultrasonic transducer that produces ultrasonic sound that may be transmitted through apertures in the translucent head plate [2].

The motor [11] may be configured to rotate, vibrate, or otherwise move the applicator disc [5] (and optional disc cover [6] if present) to stimulate the skin. For example, the motor [11] can be configured and controlled to rotate the applicator disc [5] at a particular number of revolutions per minute (RPM). In some embodiments, the RPM can be between about 50 and 300 RPM. This general range can be effective for massaging the skin and optionally for introducing an ingredient (e.g., a lotion, a salve, or another skincare substance) dispensed by the device into the skin. In some embodiments, the RPM of the motor [11] (and thus of the applicator disc [5]) can be about 150-200 RPM, which can be effective for massaging the skin without being too abrasive or disruptive.

The massaging effect of the device having a rotating applicator disc [5] can be more effective if it rotates continuously in one direction for at least several seconds or more, optionally rotating in a single direction for the entire time it is being used. This is in contrast to an oscillating circular motion, where the direction of rotation changes in shorter time intervals, e.g., on the order of about a second or less. Such oscillating motion can be more disruptive and irritating to the skin, and may potentially be abrasive and pull the skin in opposite directions at a high frequency, leading to injury or discomfort. Oscillating motion also may be less effective in introducing a lotion or other substance into the skin, e.g., by reversing the introduction of the substance when the rotation direction changes over short time intervals. In contrast, continuous rotation can be beneficial based on the smoothness of interaction with the skin as compared to an oscillating motion, and allows the contact surface to deliver a high frequency of impulses in a smooth motion.

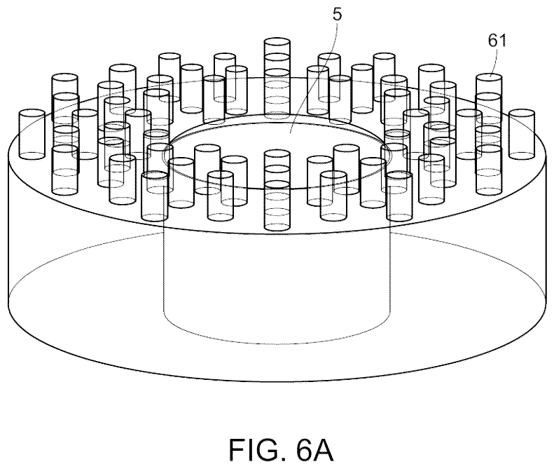

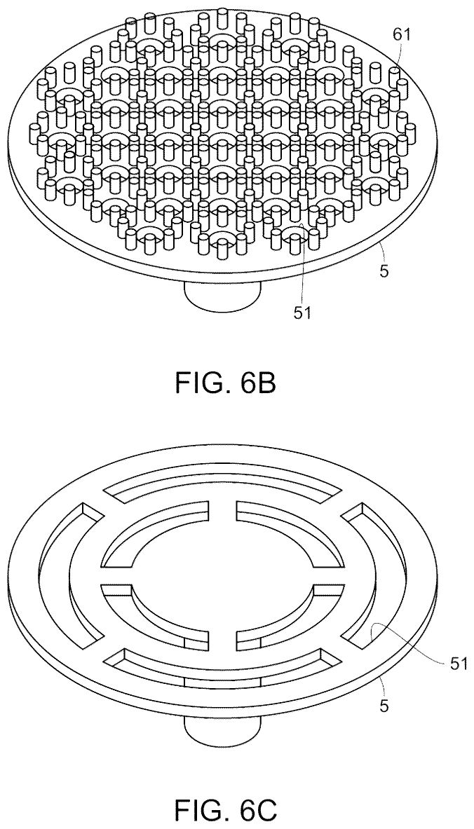

Applicator disc [5] may incorporate a variety of surface elements such as a brush with hair or tentacles, pads, abrasive material such as sand paper, silicone or rubber surfaces, metal, glass, rolling balls made from rigid materials and other constrictions where the surface interacts with the skin in a beneficial way. These protrusions or surface contours can be provided in a plurality of rotationally or radially symmetric regions on the head. FIGS. 6A-6C are top perspective views showing various examples of configurations of applicator disc [5]. For example, FIG. 6A shows an applicator disc [5] having a number of short bristles [61] provided on the contact surface. These surface elements can be non-transparent and non-translucent so long as applicator disc [5] comprises one or more transparent or translucent sections in its base through which light may shine, as shown in FIG. 6B. For example, the entire base of applicator disc [5] may transparent or translucent, and it may incorporate opaque bristles [61] or other surface elements, or it may incorporate transparent or translucent bristles or other surface elements. The applicator disc [5] may comprise non-transparent or non-translucent surface elements interspersed between transparent or translucent sections [51] of its base.

FIG. 6C shows an applicator disc [5] that includes transparent sections [51] configured to allow light from the light sources [23] to shine through. (The transparent regions [51] in FIG. 6C may appear as arc-shaped openings in the applicator disc [5], but they are actually sections of a transparent material.) In another embodiment, the transparent sections [51] in the applicator disc [5] of FIG. 6C can be curved slots that also allow light to pass therethrough. The applicator cap [5] in FIG. 6C can be useful when a removable disc cover [6] is used to contact the skin or other body part of the user, as shown in FIG. 1. For example, different surface elements may be attached to applicator disc [5] by means of a disc cover [6], for instance a disposable cover, also having substantially transparent or translucent portions, whereby light from panel [3] can shine through transparent or translucent head plate [2], transparent or translucent applicator disc [5], and disc cover [6]. In some embodiments, the disc cover [6] may comprise an abrasive or non-abrasive cleaning pad or wipe. The disc cover [6]may be made attachable by various attachment means such as snap in or snap on engagement, a screw connection, magnetic, adhesive, or hook and loop fasteners, etc. The disc cover [6] may be comprised of flexible or inflexible materials. For instance, with reference to FIG. 1 or 3, the disc cover [6] may comprise a flexible silicone cover with a lip that secures around the edge of the applicator disc [5], or an inflexible cover that snaps onto or screws over applicator disc [5].

Similar to applicator disc [5], disc cover [6] may comprise a base of one or more transparent or translucent sections such that light can emanate through head plate [2], applicator disc [5], and through said transparent or translucent sections of disc cover [6]. For example, the entire disc cover [6] may be a transparent or translucent flexible silicone cover, comprising transparent or non-transparent bristles, or it may comprise non-transparent or non-translucent surface elements interspersed between transparent or translucent sections of its base, similar to the examples shown in FIGS. 6A-6C.

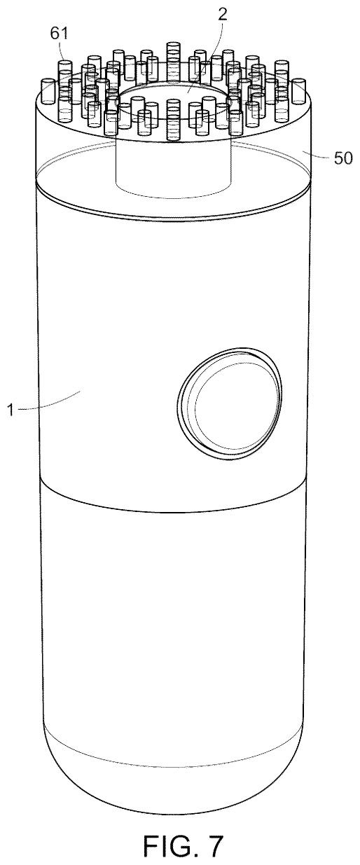

FIG. 7 is a side perspective view of a representative embodiment of the light therapy device. As another example, the applicator disc [5] may be an attachment that surrounds transparent or translucent head plate [2], wherein the applicator attachment [50] attaches to a part of the housing [1] that rotates while head plate [2], and panel [3] and its light sources [23] remain fixed. For example, said applicator attachment [50] may comprise a transparent or translucent disc section over the transparent or translucent head plate [2], or it may surround the transparent or translucent head plate [2].

In this manner, the light therapy device can be used by holding the housing [1] of the device and moving the device across the skin, hair, scalp or nails, or other applied surface in a manner where applicator disc [5] is touching or facing said skin, hair, scalp or nails, or other applied surface. The light sources [23] mounted on the panel [3] behind the applicator disc [5] shine through to provide phototherapy benefits while the device is applied. For instance, the device may be applied during facial cleansing to clean the skin while simultaneously providing phototherapy benefits. It will be noted that the device may have other applications other than to skin, as light therapy can have broad applications. As another example, the device can be used with UV light to sterilize dishes while washing. The device may be used without attaching the applicator disc [5], for instance, when light is desired to be applied without the additional effects of the applicator disc [5]. Any of the embodiments described herein may be waterproof, e.g., to facilitate their use in wet environments (e.g., in a shower, or for sterilizing dishes), and to prevent undesirable leakage of a dispensed ingredient or substance into other components of the device.

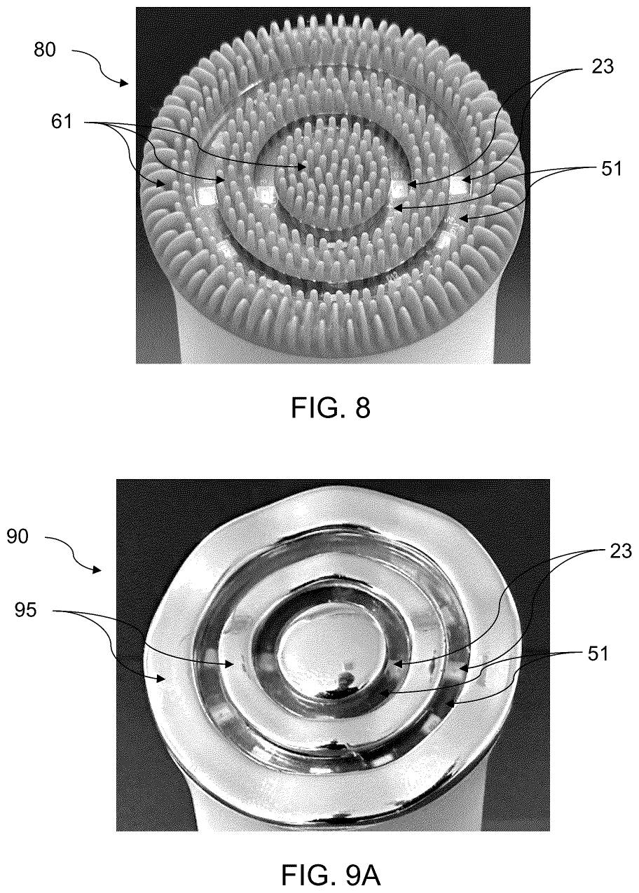

FIG. 8 shows one exemplary applicator head [80] that can be used with embodiments of the disclosure. The applicator head 80 includes concentric rings of bristles [61] having transparent ring-shaped sections [51] between them, allowing light from the light sources [23] to shine through them. The bristles [61] can be made from pliable, semi-rigid, and/or rigid materials, including silicone, rubber, polymers, or plastics. In some embodiments, the bristles [61] can have Shore A hardness values between about 20 and 80, e.g., ranging from very pliable to semi-rigid. Bristles having different shapes and/or Shore A hardness values may be combined on a single applicator head [80] to produce different effects on the skin or other surfaces. For example, small or thin bristles [61] that can be soft or rigid, shown in the inner rings of FIG. 8, can be effective for cleaning pores. Wider, softer bristles [61] that are shaped more like ribs, shown along the outer perimeter of FIG. 8, can act as a wiper blade to scrape debris from the skin and provide a massaging action. Some bristles [61] can also be provided in the form of dome-shaped bumps, which may be soft or rigid, to generate a massaging effect.

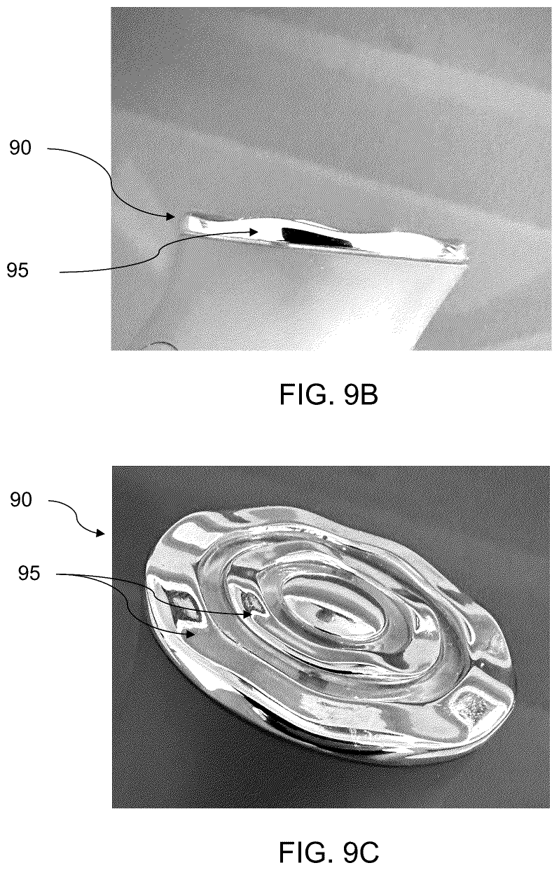

In another embodiment, shown in FIG. 9A, an applicator head [90] that can be used with embodiments of the disclosure includes concentric rings [95] formed of rigid materials, and having a wavy or scalloped surface profile. This type of applicator head [90] can be effective for massaging or rubbing the skin without much abrasion, and can also be used to rub a dispensed ingredient or substance into the skin without the substance getting trapped between bristles. For example, the hardness of the wavy rings [95] can be between about 60 and 100 on the Shore D scale. FIGS. 9B and 9C show a side view and a perspective view, respectively, of the wavy rings [95] provided on the applicator head [90] of FIG. 9A. In other embodiments, the number of wavy rings [95] provided on an applicator head [90] can be varied, e.g., between 1 and 5-6 rings. The number of waves formed on each ring [95] and the width of each ring [95] can also be varied.

The massaging effect of the applicator head [90] having wavy rings [95] can be varied or determined by the number of waves and the RPM at which the applicator head [90] is rotated. For example, in FIGS. 9A-9C, the inner ring of exemplary applicator head [90] has 4 waves, whereas the outer ring has 8 waves. Using an exemplary RPM range of 60 to 280, the total number of impulses per minute that the wave pattern will deliver for this configuration will range between 240 and 1,120 for the inner ring, and between 480 and 2,240 for the outer ring. Different numbers of waves can be provided on the wavy rings [95], and the actual number of impulses per minute induced by a particular ring [95] can be determined as the number of waves on the ring [95] multiplied by the RPM of the rotating applicator head [90]. These ranges of impulses per minute should be effective in massaging the skin and driving skincare ingredients in without injuring the users, and impulse rates somewhat higher or lower than these ranges should also be effective. In any of the embodiments described herein, the device can be configured (via, e.g., the motor [11], microcontroller [21], and/or a switch [11]) to rotate any of the described applicator discs, applicator heads, and/or disc covers at a particular RPM, a user-selectable RPM, and/or a plurality of predetermined RPMS.

The applicator head [90] can be formed of a rigid material such as a hard plastic or polymer. In other embodiments, the applicator head [90] can be formed at least in part of a metal. The entire head [90] may be formed of metal, the wavy rings [95] can be formed of a metal provided on a plastic or polymer base, or the wavy rings [95] can be formed of a plastic or polymer coated or plated with a metallic material. A metal head, whether it contains light rings or is solid without any light rings, can provide certain advantages. For example, metal wavy rings [95] can provide an effective way of delivering heat to the top surface of the applicator head [90]. Because it moves in close proximity to the brush body, heat that is delivered from the top of the brush body could be conducted through the metal wavy rings [95] and to the surface of the user's skin. Heat can be generated, e.g., by heating elements embedded in the wavy rings [95] or in the housing [1] below the applicator head [90], by heat captured off the light elements [23], or by a combination of both. Similarly, cooling effects can be achieved by provided cooling elements embedded in the wavy rings [95] and/or in the body of the applicator head [90].

Further, the metal applicator head [90] can also be detached and placed in a refrigerator or freezer, or under a stream of cold or warm water, to alter its temperature and deliver a temporary cold or warm action to the user's skin.

In still further embodiments, galvanic currents can be delivered by wavy rings [95] that are at least partially metallic to a user's skin or other body parts. Sometimes referred to as cosmetic electrotherapy, galvanic current can be used to infuse skin tissue and facial muscles with a low to medium electrical current, facilitating such positive effects as stimulating blood circulation, boosting cellular metabolism, breaking down congestion in the pores, and even providing a form of non-surgical skin tightening. Delivery of such galvanic current can be achieved by provided electrical isolation between the wavy rings [95]. A different voltage can be applied to each ring [95], e.g., using multiple brush-type electrical contacts between a voltage source in the device and the shaft [4] or a power source (such as a battery) provided on, in, or affixed to the applicator head [90]. Each ring [95] can be provided in electrical communication with a different contact on the shaft [4] to induce a different voltage on each ring [95], thereby generating galvanic currents on the user's skin between the rings [95] when the applicator head [95] is in contact with the skin. In a further embodiment, a single wavy ring [95] can have two or more electrically isolated metallic surface regions, with each region being connected to a different voltage level using contacts in the shaft [4] as described above. Such regions can be formed by providing physically separated metallic surface components mounted on a non-conductive base to form a ring [95].

In further embodiments, the applicator head [90] can be provided without transparent sections [51]. In these embodiments, the head [90] with wavy rings [95] can be used to provide any of the other benefits described above including, e.g., an effective massage effect, application of heat or cooling, application of galvanic currents, and/or application of a dispensed ingredient or substance into the skin.

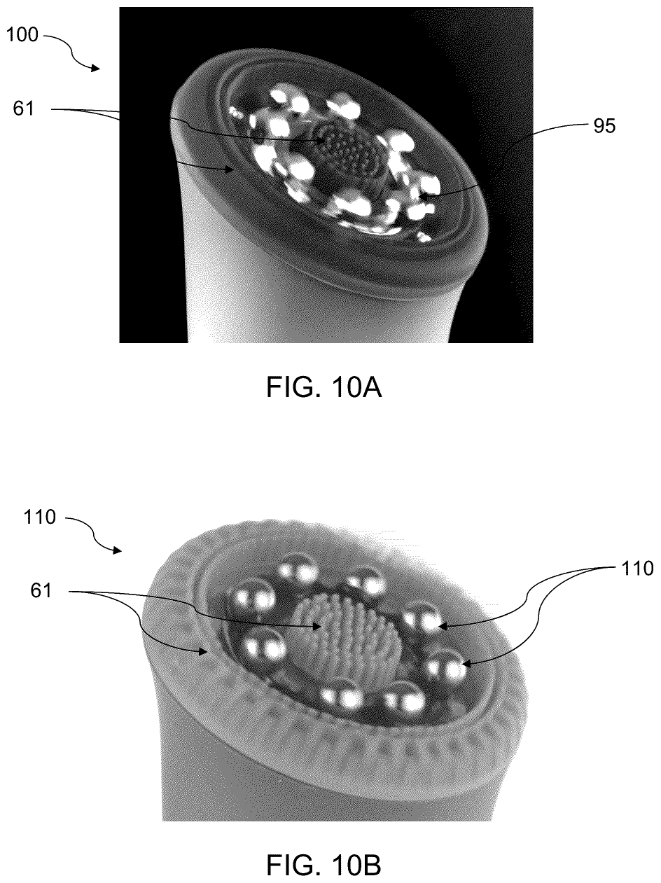

In still further embodiments, shown in FIGS. 10A and 10B, a combination of hard materials (e.g. metal) and softer materials can be provided on an applicator head. For example, the applicator head [100] in FIG. 10A includes soft bristles [61] in the central and outer regions, with a wavy ring [95] between them. The wavy ring [95] can be formed of a metal or another rigid material to provide impulses for effective massaging. The applicator head [110] in FIG. 10B also includes soft bristles [61] in the central and outer regions, with a plurality of beads [120] between them. The beads [120] can be formed of a metal or another rigid material to provide impulses for effective massaging, and can be round, ovoid, or have another rounded shape or surface profile.

The number of waves in the ring [95] and/or the number of beads [120] in FIGS. 10A and 10B, respectively, can be selected to provide effective stimulation of the skin or other body parts at the RPM or RPM range at which these heads rotate. The rigid wavy or beaded sections shown in FIGS. 10A and 10B can work effectively with the softer bristles [61] to generate impulses on the skin while also providing gentler rubbing or sweeping effects when the device is being used. The number and widths of regions of rigid material (shown as a single ring in FIGS. 10A-B) and regions of bristles [61] can also be varied depending on the desired type of interactions with the skin.

In further embodiments of the disclosure, an applicator device can be provided that may include any of the features described herein above. As used herein, the term applicator device can refer to a device that combines a container that dispenses a soluble or therapeutic substance while at the same time providing light or therapeutic light and/or mechanical stimulation (e.g., using rotating brushes or other moving contoured surfaces) that complements or enhances the treatment of the part of the skin, hair or nails of the user to which the substance is being applied.

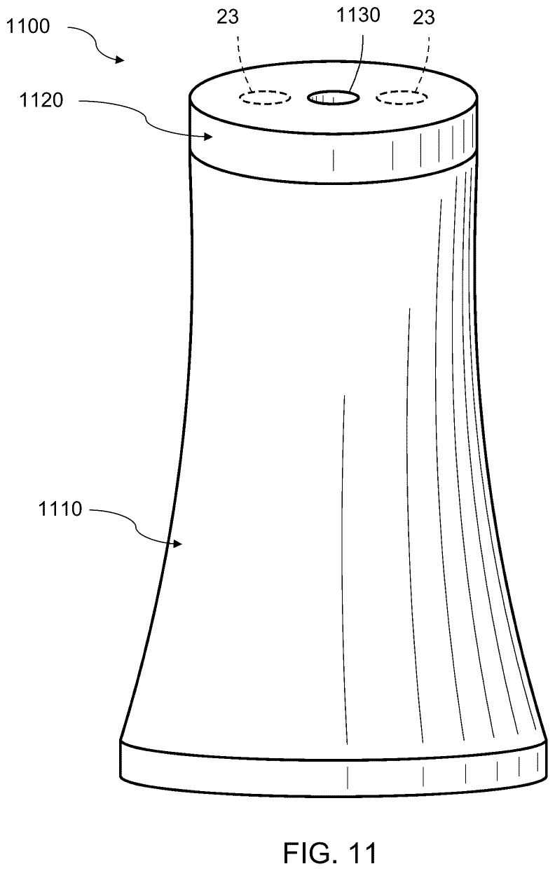

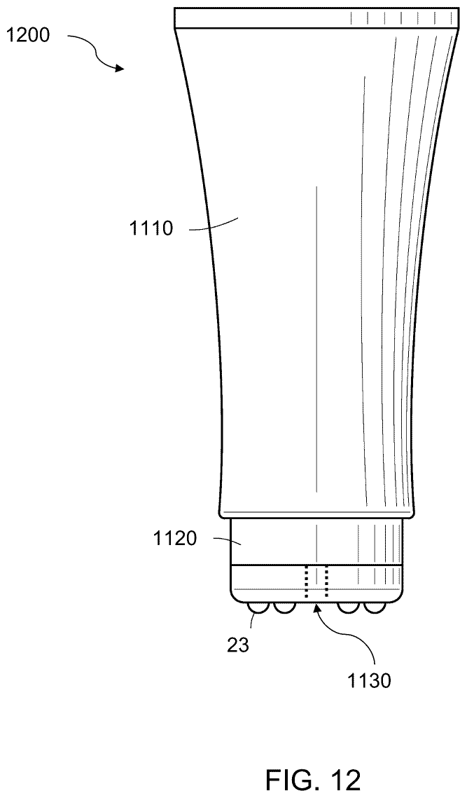

FIGS. 11-14 show various exemplary embodiments of such applicator devices or components thereof. In one aspect of the disclosed embodiments, an applicator device may include a tube or reservoir that contains a substance to be applied to the skin or other tissue, together with an applicator that contains a power source and a light source that is attached to the tube. FIG. 11 illustrates an applicator device [1100] that includes a tube [1110] that is attached to an applicator [1120] through which the ingredient inside the tube [1110] will be squeezed out via opening or cavity [1130], while the applicator [1120] also contains a light source [23], such as a laser (e.g., a diode laser) or LED light. When the user presses the applicator [1120] against their skin, hair, nails or scalp and squeezes out the ingredients inside the tube [1110], the ingredient penetrates or bonds with the user's skin, hair, scalp or nails. Simultaneously, the light source [23] reaches the ingredient as well as the user's skin, hair, scalp or nails. FIG. 12 is another view of a similar applicator device [1200] that also includes a tube [1110], an applicator [1120], an opening [1130] through which an ingredient inside the tube [1110] can be dispensed onto skin, hair, nails or scalp, and one or more light sources [23].

The light source [23] can impact the ingredient, so that chemical processes or alterations in the ingredient is activated. The light source [23] can also impact the user's skin, scalp or nails, by increasing blood flow, in the case of certain red frequencies of light, or by killing bacteria or viruses on the surface of the skin, such is the case in with certain blue frequencies of light.

The light source [23] may also act as a source of heat that melts the ingredient when dispensed through the applicator [1120] or that heats up either the ingredient, the skin, hair or nails of the user or both ingredient and body part of the user in order to provide therapeutic or cosmetic benefits.

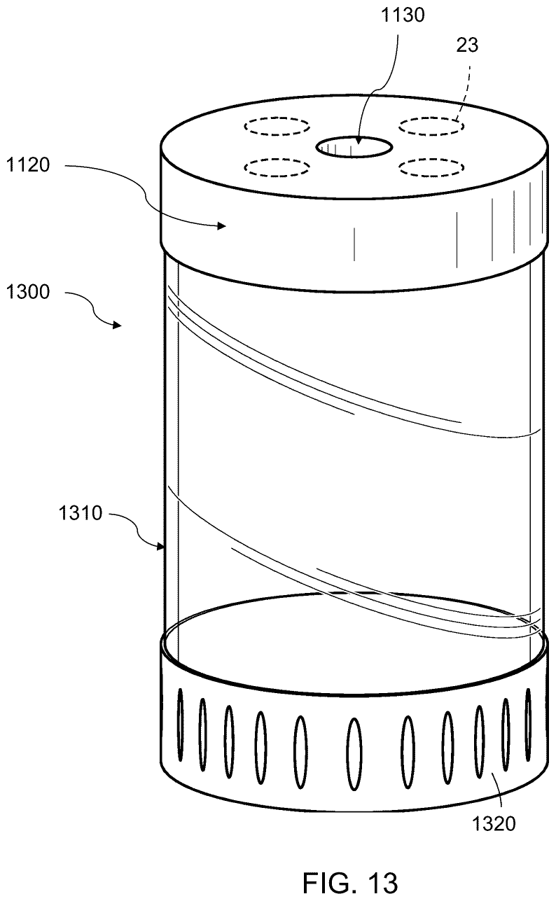

FIG. 13 shows another embodiment of an applicator device [1300] that includes a rigid or semi rigid tube [1310] with an applicator cap [1120] attached on one end, in which one or more light sources [23] are embedded or attached to, as well as a rotatable activator [1320] that will squeeze the ingredient embedded in the rigid or semi right tube [1310] out through an opening [1130] in the applicator cap [1120] when rotated.

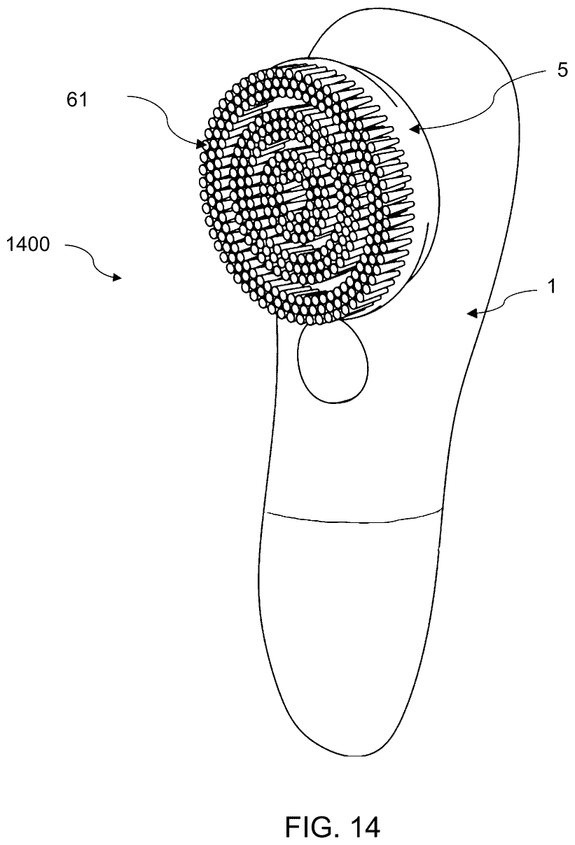

In another embodiment, the applicator can be a rotating brush or applicator surface that contains one or more sources of light [23] and that project on the user's skin, hair or nails while the rotating brush or applicator surface engages to clean, massage or provide therapeutic benefits to the user's skin, scalp, nails or hair. FIG. 14 illustrates an applicator device [1400], similar to other embodiments described herein but with an applicator disc [5] containing bristles [61] that is provided at an angle to the main body [1] of the device. The rotating applicator disc [5] or any other similar rotating element described herein can be provided at any desired angle relative to the body [1] in any of the embodiments described herein. The applicator disc [5] can rotate, while simultaneously dispensing light and ingredients to the user's skin, scalp, hair or nails.

The foregoing merely illustrates the principles of the exemplary embodiments of the present disclosure. Other variations to the exemplary embodiments can be understood and effected by those skilled in the art in practising the claimed invention from a study of the drawings, the disclosure, and the appended paragraphs. The mere fact that certain features are described in different paragraphs and/or illustrated in different figures does not indicate that any combination of these features cannot be used advantageously. Various modifications and alterations to the described exemplary embodiments will be apparent to those skilled in the art in view of the teachings herein. It will thus be appreciated that those skilled in the art will be able to devise numerous techniques which, although not explicitly described herein, embody the principles of the invention and are thus within the spirit and scope of the present disclosure. Further, all patents, patent applications, and other publications referenced, cited, or identified herein are hereby incorporated by reference herein in their entireties.

What is claimed is:

1. A light treatment device for skin and other tissues, comprising:
a housing;
a head plate rotatably affixed to the housing, wherein:
the head plate comprises a plurality of at least one of radially or rotationally symmetric regions,
each region comprises at least one of resilient bristles, rigid protrusions, or a rigid wavy surface; and
the head plate further comprises at least one transparent or translucent area provided between at least two of the regions,
a motor configured to rotate the head plate relative to the housing; and
at least one light-emitting element provided on the housing beneath the head plate and configured to emit light through the at least one transparent or translucent area of the head plate,
wherein the motor is configured to rotate the head plate in a non-oscillatory manner.

2. The device of claim 1, further comprising a chamber within the housing configured to dispense a substance through the head plate.

3. The device of claim 1, wherein the at least one light-emitting element is configured to emit light at one or more particular wavelengths or wavelength ranges within the spectrum of light.

4. The device of claim 1, wherein the head plate comprises a rotatable disc and a disc cover affixed to the rotatable disc.

5. The device of claim 1, wherein the device comprises a plurality of light-emitting elements and at least two of the plurality of light-emitting elements are configured to emit light at different particular wavelengths or wavelength ranges within the spectrum of light.

6. The device of claim 5, wherein the different particular wavelengths or wavelength ranges are user-selectable.

7. The device of claim 5, wherein the different particular wavelengths or wavelength ranges are automatically selected based on the head plate.

8. The device of claim 1, wherein the head plate comprises a plurality of at least one of radially or rotationally symmetric regions, wherein each region comprises a wavy surface that is at least partially metallic.

9. The device of claim 8, further comprising an electrical source configured to apply a different voltage to at least two electrically isolated metallic portions of the wavy surfaces.

10. The device of claim 1, wherein the head plate comprises at least two of resilient bristles, rigid protrusions, or rigid wavy surfaces.

11. The device of claim 1, further comprising a sensor configured to detect a proximity of the head plate to a surface and to shut off power to at least one of the motor or the at least one light-emitting element when the head plate is not proximal to a surface.

12. The device of claim 1, wherein the device is configured to provide heat to the head plate from at least one of the at least one light-emitting element or a resistance heating arrangement provided in the housing.

13. The device of claim 1, wherein the device is configured to provide heat to the head plate from at least one of a resistance heating arrangement provided in the head plate, or a thermal reservoir provided in the head plate.
